(19) United States
(12) Patent Application Publication
(10) Pub. No.: US 2023/0091966 A1
(43) Pub. Date: Mar. 23, 2023

(54) METHODS FOR THE TREATMENT OF CARDIOVASCULAR DISEASE WITH CYCLODEXTRINS

(71) Applicant: Beren Therapeutics, P.B.C., West Hollywood, CA (US)

(72) Inventors: Diana Kerwin, West Hollywood, CA (US); Jason Camm, West Hollywood, CA (US)

(21) Appl. No.: 17/985,762

(22) Filed: Nov. 11, 2022

Related U.S. Application Data
(63) Continuation of application No. PCT/US21/48084, filed on Aug. 27, 2021.
(60) Provisional application No. 63/071,257, filed on Aug. 27, 2020.

(51) Int. Cl. A61K 31/724; A61P 9/10

(52) U.S. Cl. CPC A61K 31/724; A61P 9/10

(57) ABSTRACT

Disclosed herein are methods treating atherosclerosis and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease) by administering a therapeutically effective amount of 2-hydroxypropyl-beta-cyclodextrin to the subject.

### CROSS-REFERENCE TO RELATED APPLICATIONS

This application is a continuation of and claims priority to the International Patent Application No. PCT/US2021/048084, filed Aug. 27, 2021 and entitled “Methods for the Treatment of Cardiovascular Disease with Cyclodextrins,” which claims the benefit of and priority to U.S. Provisional Application No. 63/071,257, filed Aug. 27, 2020, the entire contents of which are herein incorporated by reference in their entirety and for all purposes.

### BACKGROUND

Cardiovascular disease is a class of diseases that involve the heart and blood vessels. Atherosclerosis is thought to be a major cause of cardiovascular disease. Atherosclerosis, or the narrowing and hardening of arteries caused by a buildup of plaque, can lead to, e.g., heart attacks (myocardial infarction), strokes, peripheral vascular disease, and coronary artery disease. Atherosclerosis is a heavy burden to our modern society. Preclinical data suggest 2-hydroxypropyl -beta-cyclodextrins could have profound beneficial effects on the pathomechanisms responsible for atherosclerotic disease development and arrest or reverse the progression of cardiovascular disease. Therefore, 2-hydroxypropyl-beta-cyclodextrins may provide a novel treatment option for cardiovascular disease.

### SUMMARY OF THE DISCLOSURE

There is a need for safe and effective treatments for cardiovascular disease. This disclosure addresses this unmet need.

In one aspect, a method of treating atherosclerosis and/or atherosclerotic cardiovascular disease in a human individual is provided, the method comprising: administering a therapeutically effective amount of 2-hydroxypropyl-beta-cyclodextrin to the human individual, the therapeutically effective amount of 2-hydroxypropyl-beta-cyclodextrin being: (a) an amount effective to increase a circulating and/or systemic level of one or more oxysterol in the human individual by at least about 10% after the administering as compared to prior to the administering; (b) an amount effective to increase plasma cholesterol crystal dissolution capacity (CCDC) by at least about 10% after the administering as compared to prior to the administering; (c) an amount effective to increase a level of ABCA1 and/or ABCG1 by at least about 10% after the administering as compared to prior to the administering; (d) about 50 mg/kg to about 2,000 mg/kg; or (e) any combination thereof, thereby treating atherosclerosis and/or atherosclerotic cardiovascular disease in the human individual. In some cases, the atherosclerotic cardiovascular disease is selected from the group consisting of: coronary artery disease (CAD), peripheral artery disease (PAD), and peripheral vascular disease (PVD).

In another aspect, a method of reducing or inhibiting the development of cholesterol rich plaque in a human individual is provided, the method comprising administering a therapeutically effective amount of 2-hydroxypropyl-beta-cyclodextrin to the human individual, the therapeutically effective amount being: (a) an amount effective to increase a circulating and/or systemic level of one or more oxysterol in the human individual by at least about 10% after the administering as compared to prior to the administering; (b) an amount effective to increase plasma cholesterol crystal dissolution capacity (CCDC) by at least about 10% after the administering as compared to prior to the administering; (c) an amount effective to increase a level of ABCA1 and/or ABCG1 by at least about 10% after the administering as compared to prior to the administering; (d) about 50 mg/kg to about 2,000 mg/kg; or (e) any combination thereof, thereby reducing or inhibiting the development of cholesterol rich plaque in the human individual.

In some cases, the therapeutically effective amount is about 4 g to about 250 g of the 2-hydroxypropyl -beta-cyclodextrin. In some cases, the therapeutically effective amount is an amount sufficient to achieve a serum, plasma, and/or whole blood concentration of 2-hydroxpropyl -beta-cyclodextrin of about 0.6 mM to about 3 mM. In some cases, the therapeutically effective amount is an amount effective to increase a circulating and/or systemic level of one or more oxysterol in the human individual by at least about 10% after the administering as compared to prior to the administering. In some cases, the circulating and/or systemic levels comprise serum, plasma, and/or whole blood levels. In some cases, the one or more oxysterols are selected from the group consisting of: 27-hydroxycholesterol and 24-hydroxycholesterol. In some cases, the at least about 10% comprises at least about 15%, at least about 20%, at least about 30%, at least about 40%, or at least about 50%. In some cases, the therapeutically effective amount is an amount effective to increase a circulating and/or systemic level of one or more oxysterol to about 40 ng/mL or greater. In some cases, the therapeutically effective amount is an amount effective to increase a circulating and/or systemic level of one or more oxysterol to at least about 40 ng per mg of total circulating and/or systemic cholesterol. In some cases, the one or more oxysterol comprises 27-hydroxycholesterol. In some cases, the therapeutically effective amount is an amount effective to increase the circulating and/or systemic level of 27-hydroxycholesterol to at least about 100 ng/mL. In some cases, the therapeutically effective amount is an amount effective to increase the circulating and/or systemic level of 27-hydroxycholesterol to at least about 90 ng per mg of total circulating and/or systemic cholesterol. In some cases, the therapeutically effective amount is an amount sufficient to sustain the circulating and/or systemic level of the one or more oxysterol for at least 24 hours. In some cases, the therapeutically effective amount is an amount effective to increase plasma cholesterol crystal dissolution capacity (CCDC) by at least about 10% after the administering as compared to prior to the administering. In some cases, the therapeutically effective amount is an amount effective to increase a level of ABCA1 and/or ABCG1 by at least about 10% at after the administering as compared to prior to the administering. In some cases, the therapeutically effective amount is about 50 mg/kg to about 2,000 mg/kg. In some cases, the therapeutically effective amount is at least about 100 mg/kg. In some cases, the therapeutically effective amount is at least about 250 mg/kg. In some cases, the therapeutically effective amount is at least about 500 mg/kg. In some cases, the therapeutically effective amount is at least about 1,000 mg/kg. In some cases, the therapeutically effective amount is at least about 1,500 mg/kg. In some cases, the therapeutically effective amount is about 500 mg/kg to about 1,500 mg/kg. In some cases, the therapeutically effective amount is about 800 mg/kg to about 1,200 mg/kg. In some cases, the human individual is at least 30 years old. In some cases, the administering further comprises: (i) administering, at a first time point, a therapeutically effective first dose of 2-hydroxypropyl-beta-cyclodextrin to the human individual; and (ii) administering, at a second time point, a therapeutically effective second dose of 2-hydroxypropyl-beta-cyclodextrin to the human individual. In some cases, the second time point is at least 1 week after the first time point. In some cases, the second time point is at least 2 weeks after the first time point. In some cases, the second time point is at least one month after the first time point. In some cases, the treating comprises decreasing or preventing progression and/or development of atherosclerosis in the human individual. In some cases, the treating comprises mediating the regression of atherosclerotic plaques in the human individual. In some cases, the treating results in one or more of the following: a) liver enzyme (e.g., ALT, AST) levels less than 2.5 times to normal; b) serum creatinine levels less than 0.3 mg/dl; or c) no substantial loss of sensorineural hearing. In some cases, the administering is by intravenous administration.

In another aspect, a pharmaceutical composition is provided comprising: an amount of 2-hydroxypropyl -beta-cyclodextrin effective to treat atherosclerosis and/or atherosclerotic cardiovascular disease in a human individual, and a pharmaceutically acceptable excipient. In yet another aspect, a pharmaceutical composition is provided comprising: about 4 g to about 250 g of 2-hydroxypropyl-beta-cyclodextrin and a pharmaceutically acceptable excipient. In some cases, the amount of 2-hydroxypropyl-beta-cyclodextrin is an amount effective to increase a circulating and/or systemic level of one or more oxysterol in the human individual by at least about 10% after administering the pharmaceutical composition to the human individual. In some cases, the amount of 2-hydroxypropyl-beta-cyclodextrin is an amount effective to increase plasma cholesterol crystal dissolution capacity (CCDC) by at least about 10% after administering the pharmaceutical composition to the human individual. In some cases, the amount of 2-hydroxypropyl-beta-cyclodextrin is an amount effective to increase a level of ABCA1 and/or ABCG1 by at least about 10% after administering the pharmaceutical composition to the human individual. In some cases, the pharmaceutical composition is formulated for single dose administration. In some cases, the pharmaceutical composition is formulated for intravenous administration.

In yet another aspect, a kit is provided comprising: (a) one or more container; and (b) the pharmaceutical composition of any one of the preceding, wherein the pharmaceutical composition is contained within the one or more container. In some cases, the kit further comprises (c) instructions for use of the pharmaceutical composition for the treatment of atherosclerosis and/or atherosclerotic cardiovascular disease in a human individual and/or reducing or inhibiting the development of cholesterol rich plaque in a human individual. In some cases, at least one of the one or more container is an IV infusion bag. In some cases, the one or more container comprises a single container comprising the pharmaceutical composition and one or more additional active pharmaceutical ingredients. In some cases, the one or more container comprises a first container containing the pharmaceutical composition and a second container containing one or more additional active pharmaceutical ingredients. In some cases, the kit further comprises one or more additional components selected from the group consisting of: an IV infusion bag, a catheter, tubing, a needle, a syringe, a solution, and any combination thereof.

In another aspect, a method of treating atherosclerosis and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease) in a human is provided, the method comprising: administering a therapeutically effective amount of 2-hydroxypropyl-beta-cyclodextrin to the human, the therapeutically effective amount being an amount effective to increase a circulating and/or systemic (e.g., serum, plasma, and/or whole blood) level of one or more oxysterol (e.g., 27-hydroxycholesterol, 24-hydroxycholesterol) by at least about 10% (e.g., at least about 15%, at least about 20%, at least about 30%, at least about 40%, at least about 50%) as compared to baseline, thereby treating atherosclerosis and/or atherosclerotic cardiovascular disease in the human.

In another aspect, a method of reducing or inhibiting the development of cholesterol rich plaque in a human is provided, the method comprising administering a therapeutically effective amount of 2-hydroxypropyl-beta-cyclodextrin to the human, the therapeutically effective amount being an amount effective to increase a circulating and/or systemic (e.g., serum, plasma, and/or whole blood) level of one or more oxysterol (e.g., 27-hydroxycholesterol, 24-hydroxycholesterol) by at least about 10% (e.g., at least about 15%, at least about 20%, at least about 30%, at least about 40%, at least about 50%) as compared to baseline, thereby reducing or inhibiting the development of cholesterol rich plaque in the human.

In another aspect, a method of treating atherosclerosis and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease) in a human is provided, the method comprising: administering a therapeutically effective amount of 2-hydroxypropyl-beta-cyclodextrin to the human, the therapeutically effective amount being an amount effective to increase a circulating and/or systemic (e.g., serum, plasma, and/or whole blood) level of one or more oxysterol (e.g., 27-hydroxycholesterol, 24-hydroxycholesterol) to about 40 ng/ml or greater, thereby treating atherosclerosis and/or atherosclerotic cardiovascular disease in the human.

In yet another aspect, a method of treating atherosclerosis and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease) in a human is provided, the method comprising: administering a therapeutically effective amount of 2-hydroxypropyl-beta-cyclodextrin to the human, the therapeutically effective amount being an amount effective to increase a circulating and/or systemic (e.g., serum, plasma, and/or whole blood) level of one or more oxysterol (e.g., 27-hydroxycholesterol, 24-hydroxycholesterol) to at least 40 ng per mg of total circulating and/or systemic cholesterol, thereby treating atherosclerosis and/or atherosclerotic cardiovascular disease in the human.

In any of the preceding aspects, the therapeutically effective amount is from about 50 mg/kg to about 2,000 mg/kg (or from about 4 g to about 250 g). In any of the preceding aspects, the therapeutically effective amount is an amount sufficient to achieve a serum, plasma, and/or whole blood concentration of 2-hydroxypropyl-beta-cyclodextrin of about 0.6 mM to about 3 mM.

In another aspect, a method of treating atherosclerosis and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease) in an individual is provided, the method comprising: administering a therapeutically effective amount of 2-hydroxypropyl-beta-cyclodextrin to the individual, the therapeutically effective amount being from about 50 mg/kg to about 2,000 mg/kg (or from about 4 g to about 250 g), thereby treating atherosclerosis and/or atherosclerotic cardiovascular disease in the individual.

In yet another aspect, a method of treating atherosclerosis and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease) in an individual is provided, the method comprising: administering a therapeutically effective amount of 2-hydroxypropyl-beta-cyclodextrin to the individual, the therapeutically effective amount being an amount sufficient to achieve a serum, plasma, and/or whole blood concentration of 2-hydroxypropyl-beta-cyclodextrin of about 0.6 mM to about 3 mM, thereby treating atherosclerosis and/or atherosclerotic cardiovascular disease in the individual.

In any of the preceding aspects, the therapeutically effective amount is an amount effective to increase a circulating and/or systemic (e.g., serum, plasma, and/or whole blood) level of one or more oxysterol (e.g., 27-hydroxycholesterol, 24-hydroxycholesterol) by at least about 10% (e.g., at least about 15%, at least about 20%, at least about 30%, at least about 40%, at least about 50%) as compared to baseline. In any of the preceding aspects, the therapeutically effective amount is an amount effective to increase a circulating and/or systemic (e.g., serum, plasma, and/or whole blood) level of one or more oxysterol (e.g., 27-hydroxycholesterol, 24-hydroxycholesterol) to about 40 ng/ml or greater. In any of the preceding aspects, the therapeutically effective amount is an amount effective to increase a circulating and/or systemic (e.g., serum, plasma, and/or whole blood) level of one or more oxysterol (e.g., 27-hydroxycholesterol, 24-hydroxycholesterol) to at least about 40 ng per mg of total circulating and/or systemic cholesterol. In any of the preceding aspects, the oxysterol is 27-hydroxycholesterol. In any of the preceding aspects, the therapeutically effective amount is an amount effective to increase the circulating and/or systemic (e.g., serum, plasma, and/or whole blood) level of 27-hydroxycholesterol to at least about 100 ng/ml. In any of the preceding aspects, the therapeutically effective amount is an amount effective to increase the circulating and/or systemic level (e.g., serum, plasma, and/or whole blood) level of 27-hydroxycholesterol to at least about 90 ng per mg of total circulating and/or systemic cholesterol. In any of the preceding aspects, the oxysterol is 24-hydroxycholesterol. In any of the preceding aspects, the therapeutically effective amount is an amount effective to increase the circulating and/or systemic (e.g., serum, plasma, and/or whole blood) level of 24-hydroxycholesterol to at least about 50 ng/ml. In any of the preceding aspects, the therapeutically effective amount is an amount effective to increase the circulating and/or systemic (e.g., serum, plasma, and/or whole blood) level of 24-hydroxycholesterol to at least about 40 ng per mg of total circulating and/or systemic cholesterol. In any of the preceding aspects, the therapeutically effective amount is an amount sufficient to sustain the circulating and/or systemic level of the one or more oxysterol for at least 24 hours (e.g., at least 48 hours, at least 72 hours). In any of the preceding aspects, the therapeutically effective amount is at least about 100 mg/kg. In any of the preceding aspects, the therapeutically effective amount is at least about 250 mg/kg. In any of the preceding aspects, the therapeutically effective amount is at least about 500 mg/kg. In any of the preceding aspects, the therapeutically effective amount is at least about 1,000 mg/kg. In any of the preceding aspects, the therapeutically effective amount is at least about 1,500 mg/kg. In any of the preceding aspects, the therapeutically effective amount is from about 500 mg/kg to about 1,500 mg/kg. In any of the preceding aspects, the therapeutically effective amount is from about 800 mg/kg to about 1,200 mg/kg. In any of the preceding aspects, the human or individual is at least 30 (e.g., at least 40, at least 50, at least 60, at least 70, at least 80, at least 90) years old. In any of the preceding aspects, the therapeutically effective amount is an amount effective to increase a circulating and/or systemic (e.g., serum, plasma, and/or whole blood) level of one or more oxysterol (e.g., 27-hydroxycholesterol) by at least about 10% (e.g., at least about 15%, at least about 20%, at least about 30%, at least about 40%, at least about 50%) as compared to baseline for at least 72 hours. In any of the preceding aspects, the administering further comprises: (i) administering, at a first time point, a therapeutically effective first dose of 2-hydroxypropyl-beta-cyclodextrin to the human or individual; and (ii) administering, at a second time point, a therapeutically effective second dose of 2-hydroxypropyl-beta-cyclodextrin to the human or individual. In any of the preceding aspects, the second time point is at least 1 week after the first time point. In any of the preceding aspects, the second time point is at least 2 weeks after the first time point. In any of the preceding aspects, the second time point is at least one month after the first time point. In any of the preceding aspects, the second time point is determined based on 27-hydroxycholesterol levels, 24S-hydroxycholesterol levels, transcription levels of ABCA1, transcription levels of ABCG1, epigenetic (chromatin) signature of PBMC, triglyceride levels, total cholesterol levels, vLDL/LDL/HDL levels, serum or plasma cholesterol crystal dissolution, levels of pro-inflammatory mediators (e.g., interleukin-1b (IL-1b), interleukin-6 (IL-6), interleukin-18 (IL-18)). In any of the preceding aspects, the treating comprises reducing the size of atherosclerotic plaques by at least about 0.5%. In any of the preceding aspects, the treating comprises decreasing or preventing progression and/or development of atherosclerosis in the human subject. In any of the preceding aspects, the treating comprises mediating the regression of atherosclerotic plaques in the human subject. In any of the preceding aspects, the treating results in one or more of the following: a) an increase in flow-mediated vasodilation (FMD) of the brachial artery, as measured by ischemia-induced endothelial-dependent vasodilation after 7 days, using high resolution ultrasonography; b) an increase in sterol and/or oxysterol levels (e.g., 24S-hydroxy-cholesterol, 25-hydroxy-cholesterol, 27-hydroxycholesterol) in serum, plasma, urine, stool, or any combination thereof; c) an increase in cholesterol crystal dissolution as measured by a cholesterol crystal dissolution capacity assay; d) an increase in gene expression of liver X receptor (LXR)-regulated genes (e.g., ABCA1, ABCG1) in peripheral blood mononuclear cells (PBMCs); e) an increase in phagocytic activity of PBMCs; f) a decrease in a level of triglycerides and/or LDL-cholesterol; and/or an increase in a level of HDL-cholesterol and/or ApoA1 in serum, urine, stool, or any combination thereof; g) a decrease in serum markers of inflammation and myocardial damage (e.g., IL-1beta, IL-1a, IL-6, hsCRP, Troponin, CK, CK-MB, NT-pro-BNP) and/or an increase in serum levels of anti-inflammatory cytokines (e.g., IL-1ra); h) a decrease in complement activation; i) a decrease in mortality and/or all-cause mortality (ACM); j) a decrease in myocardial infarction; k) a decrease in major or minor stroke; l) a decrease in blood pressure; m) a decrease in major adverse cardiovascular events (MACE); and n) improved erectile dysfunction, in correlation to CAD severity. In any of the preceding aspects, the treating results in one or more of the following: a) liver enzyme (e.g., ALT, AST) levels less than 2.5 times to normal; b) serum creatinine levels less than 0.3 mg/dl; or c) no substantial loss of sensorineural hearing. In any of the preceding aspects, the administering is by intravenous administration.

In another aspect, a pharmaceutical composition is provided comprising: an amount of 2-hydroxypropyl -beta-cyclodextrin effective to treat atherosclerosis in a human and/or atherosclerotic cardiovascular disease (e.g., coronary artery disease (CAD), peripheral artery disease (PAD), peripheral vascular disease (PVD), stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, post-surgical or iatrogenic arterial disease); and an excipient.

In another aspect, a pharmaceutical composition is provided comprising: about 4 g to about 250 g of 2-hydroxypropyl-beta-cyclodextrin; and an excipient.

In any of the preceding aspects, the 2-hydroxypropyl-beta-cyclodextrin is sufficient to increase a circulating and/or systemic (e.g., serum, plasma, and/or whole blood) level of one or more oxysterol (e.g., 27-hydroxycholesterol) by at least about 10% (e.g., at least about 15%, at least about 20%, at least about 30%, at least about 40%, at least about 50%) as compared to baseline in a human for at least 72 hours after treatment. In any of the preceding aspects, the pharmaceutical composition is formulated for single dose administration. In any of the preceding aspects, the pharmaceutical composition is formulated for intravenous administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A, 1B, 2A, and 2B depict a non-limiting example of administration details of a placebo and 2-hydroxypropyl-beta-cyclodextrin in a Phase 1b and Phase 2a clinical trial.

FIGS. 3A and 3B depict a non-limiting example of cholesterol crystal dissolution capacity (CCDC) of plasma obtained from a male human subject treated with increasing dosages of 2-hydroxypropyl-beta-cyclodextrin.

FIGS. 4A-4C depict a non-limiting example of oxysterol levels in plasma obtained from a male human subject treated with increasing dosages of 2-hydroxypropyl-beta-cyclodextrin.

FIGS. 5A-5D depict a non-limiting example of mRNA levels of LXR transcription factor-regulated genes in a male human subject treated with increasing dosages of 2-hydroxypropyl -beta-cyclodextrin.

### DETAILED DESCRIPTION OF THE DISCLOSURE

Disclosed herein are methods for the treatment of cardiovascular disease. In some cases, the cardiovascular disease is atherosclerotic cardiovascular disease (e.g., cardiovascular disease caused or contributed to by atherosclerosis). The atherosclerotic cardiovascular disease may be any one of coronary artery disease (CAD), stroke, peripheral artery disease (PAD), peripheral vascular diseases (PVD), chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, and/or post-surgical or iatrogenic arterial disease. In some cases, PAD includes lower extremity arterial disease. In some cases, the methods involve treating and/or preventing atherosclerosis. In some cases, the methods involve treating a subject who has, is suspected of having, or is at risk of developing acute coronary syndrome (ACS) or chronic coronary syndrome (CCS) (e.g., as defined by the European Society of Cardiology). In some aspects, the methods may involve administering a therapeutically effective amount of a cyclodextrin to a subject in need thereof (e.g., a subject having, suspected of having, or at risk of developing cardiovascular disease (e.g., atherosclerotic cardiovascular disease)). In some cases, the therapeutically effective amount is an amount effective to increase a circulating and/or systemic level of one or more sterol and/or oxysterol in the subject compared to a baseline (e.g., pre-treatment with cyclodextrins). In a particular aspect, the cyclodextrin is 2-hydroxypropyl-beta-cyclodextrin.

In some embodiments, disclosed herein are methods for reducing the amount of and/or the size of, and/or changing the shape of circulating (e.g., blood, plasma, serum) cholesterol crystals (and/or clots comprising cholesterol crystals) in an individual (e.g., a human). In some cases, the methods involve treating cholesterol crystal embolization (CCE) (e.g., by preventing the occlusion of small blood vessels by, e.g., cholesterol crystal emboli, cholesterol crystal clots, cholesterol crystal/protein clots, cholesterol crystal/DNA clots (e.g., extracellular traps), etc.). In some cases, the methods involve treating one or more symptom and/or clinical manifestation of CCE. In some cases, the methods involve treating ischemia to various organs and/or tissues caused by, e.g., CCE. Generally, the methods provided herein involve administering a therapeutically effective amount of a cyclodextrin to a subject in need thereof (e.g., a subject having elevated levels of circulating cholesterol crystals (and/or clots comprising cholesterol crystals)). In a particular aspect, the cyclodextrin is 2-hydroxypropyl-beta-cyclodextrin.

In some embodiments, disclosed herein are methods for preventing or reducing the risk of developing circulating (e.g., blood, plasma, serum) cholesterol crystals (and/or clots comprising cholesterol crystals) in an individual (e.g., a human). Further disclosed herein are methods for preventing or reducing the risk of an increase in the amount of and/or size of, and/or changing the shape of, circulating (e.g., blood, plasma, serum) cholesterol crystals (and/or clots comprising cholesterol crystals) in an individual (e.g., a human). In some cases, the methods involve preventing or reducing the risk of developing of cholesterol crystal embolization (CCE) (e.g., by preventing or reducing the risk of the occlusion of small blood vessels by, e.g., cholesterol crystal emboli, cholesterol crystal clots, cholesterol crystal/protein clots, cholesterol crystal/DNA clots (e.g., extracellular traps), etc.). In some cases, the methods involve preventing or reducing the risk of developing a symptom and/or a clinical manifestation of CCE. In some cases, the methods involve preventing or reducing the risk of ischemia to various organs and/or tissues caused by, e.g., CCE (and/or a symptom or clinical manifestation thereof, e.g., renal injury, atheroembolic renal disease (ARD)). Generally, the methods provided herein involve administering a therapeutically effective amount of a cyclodextrin to a subject in need thereof (e.g., prophylactically, e.g., to a subject at risk of developing CCE). In a particular aspect, the cyclodextrin is 2-hydroxypropyl-beta-cyclodextrin.

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “about” a number refers to that number plus or minus 10% of that number. The term “about” a range refers to that range minus 10% of its lowest value and plus 10% of its greatest value.

As used herein, the terms “subject,” “individual”, and “patient” are used interchangeably. None of the terms are to be interpreted as requiring the supervision of a medical professional (e.g., a doctor, nurse, physician's assistant, orderly, hospice worker). As used herein, the subject may be any animal, including mammals (e.g., a human or non-human animal) and non-mammals. In one embodiment, the subject is a human.

As used herein, the terms “treat,” “treating”, or “treatment,” and other grammatical equivalents, include ameliorating or preventing the underlying causes of one or more symptoms of a disease or condition; alleviating, abating, or ameliorating one or more symptoms of a disease or condition; ameliorating, preventing, or reducing the appearance, severity, or frequency of one or more symptoms of a disease or condition; inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. Methods of treatment as disclosed herein include disclosures of the use of the (e.g., pharmaceutical) compositions provided herein for the treatment of any indication described herein, and include disclosures of the (e.g., pharmaceutical) compositions provided herein for the use in treating any indication described herein.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier, or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

“Pharmaceutically acceptable excipient” as used herein, refers to any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

The terms “effective amount” or “therapeutically effective amount,” as used herein, refer to a sufficient amount of an agent or a compound being administered which relieves, to some extent, one or more of the symptoms of the disease or condition being treated, or reduces the underlying cause of the disease or condition being treated. In some embodiments, the result is a reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms or underlying cause of the disease (e.g., without undue adverse side effects). In some embodiments, an appropriate “effective amount” in any individual case is determined using techniques, such as a dose escalation study. The term “therapeutically effective amount” includes, for example, a prophylactically effective amount. An “effective amount” of a compound disclosed herein may be an amount effective to achieve a desired effect or therapeutic improvement (e.g., without undue adverse side effects). An “effective amount” of a compound disclosed herein may be an amount effective to achieve one or more desired outcomes (e.g., a systemic and/or circulating level of a sterol or an oxysterol as described herein). It should be understood that, in some cases, “an effective amount” or “a therapeutically effective amount” varies from subject to subject, due to variation in metabolism of the composition, age, weight, general condition of the subject, concomitant medications the subject may be taking, the condition being treated, the severity of the condition being treated, and the judgment of the prescribing physician. In some instances, the disease or condition being treated is atherosclerotic cardiovascular disease (e.g., coronary artery disease (CAD)), or consequential diseases caused by atherosclerotic cardiovascular disease. In some cases, the disease or condition being treated is atherosclerosis. In some instances, the methods may be used to treat and/or prevent an underlying cause of atherosclerotic cardiovascular disease (e.g., CAD). The underlying cause of atherosclerotic cardiovascular disease may be, e.g., early vascular alterations (e.g., stiffening, impairments), calcification, plaque accumulation in the arteries and/or veins, elevation in circulating levels of lipids, inflammation, cholesterol accumulation, plaque formation, vessel occlusion, plaque rupture, and/or ischemia.

Methods of Treating Atherosclerotic Cardiovascular Disease

Examples 2-4 herein depict data demonstrating increased plasma cholesterol crystal dissolution capacity, increased oxysterol levels, and increased mRNA levels of the LXR transcription factor-regulated genes ABCA1 and ABCG1 in a human subject treated with 2-hydroxypropyl -beta-cyclodextrin. The data suggests that 2-hydroxypropyl-beta-cyclodextrin may be used to treat atherosclerosis and/or atherosclerotic cardiovascular disease, as described herein.

Disclosed herein are methods for treating a subject having, suspected of having, or at risk of developing atherosclerotic cardiovascular diseases. The atherosclerotic cardiovascular disease may be, e.g., CAD, PAD, PVD, stroke, chronic kidney disease (CKD) caused or contributed to by atherosclerosis, end-stage kidney disease (ESKD) caused or contributed to by atherosclerosis, acute kidney failure caused or contributed to by atherosclerosis, atherosclerotic renovascular disease (ARVD), renal artery stenosis, aortic aneurysm, idiopathic peripheral atrial hypertension, erectile dysfunction, intermittent claudication, and/or post-surgical or iatrogenic arterial disease. Further disclosed herein are methods for treating a subject having, suspected of having, or at risk of developing atherosclerosis. In some cases, the methods involve treating a subject having, suspected of having, or at risk of developing acute coronary syndrome (ACS) or chronic coronary syndrome (CCS) (e.g., as defined by the European Society of Cardiology). In some cases, treating a subject as described herein may inhibit, prevent, or reduce the development of atherosclerotic plaques (e.g., cholesterol-rich plaques and/or lipid-rich plaques) in the subject. In some cases, treating a subject as described herein may mediate, promote, enhance, or increase the regression of already-developed atherosclerotic plaques (e.g., cholesterol-rich plaques and/or lipid-rich plaques). In some cases, treating a subject as described herein may result in fewer atherosclerotic plaques in the subject, smaller atherosclerotic plaques in the subject, or both. In some cases, reducing the number and/or size of atherosclerotic plaques in the subject may ameliorate, prevent, or reduce one or more symptoms associated with atherosclerosis and/or atherosclerotic cardiovascular disease (e.g., CAD).

In various aspects, the methods involve administering a cyclodextrin to a subject. Cyclodextrins are a family of cyclic oligosaccharides, consisting of a cyclic (e.g., macrocyclic) ring of glucose subunits joined by α-1,4 glycosidic bonds. Cyclodextrins contain a number of glucose monomers in a ring formation. Common cyclodextrins include alpha-cyclodextrins (consisting of six glucose monomers), beta-cyclodextrins (consisting of seven glucose monomers), gamma-cyclodextrins (consisting of eight glucose monomers), and delta-cyclodextrins (consisting of nine glucose monomers). The outer portion of the ring structure is hydrophilic and the inner cavity of the ring structure is hydrophobic; thus, cyclodextrins generally are water soluble (e.g., due to the hydrophilic exterior), and capable of incorporating hydrophobic molecules in the cavity (e.g., due to the hydrophobic cavity). Parent cyclodextrins have limited water solubility; therefore, several chemically modified cyclodextrins have been synthesized where the hydroxyl groups are substituted with other chemical moieties to increase solubility. In various aspects, the methods provided herein involve administering a cyclodextrin to a subject (e.g., a human) in need thereof (e.g., having, suspected of having, or at risk of developing atherosclerosis and/or atherosclerotic cardiovascular disease (e.g., CAD)). In some cases, the subject has, is suspected of having, or is at risk of developing atherosclerotic plaques (e.g., cholesterol-rich plaques and/or lipid-rich plaques).

In particular embodiments, the cyclodextrin is 2-hydroxypropyl-beta-cyclodextrin. In some instances, the 2-hydroxypropyl-beta-cyclodextrin is selected from the group consisting of: Kleptose® HP Parenteral Grade (Roquette Frères, #346114; accessible at roquette.com/-/media/roquette -sharepoint-libraries/sdol_product-specification-sheet/roquette_quality_specification -sheet_kleptose-hp-parenteral-grade_50_346114_en.pdf as of Aug. 26, 2020), Kleptose® HPB Parenteral Grade (Roquette Frères, #346111; accessible at roquette.com/-/media/roquette-sharepoint-libraries/sdol_product-specification-sheet/roquette _quality_specification-sheet_kleptose-hpb-parenteral-grade_50_346111_en.pdf as of Aug. 26, 2020), Kleptose® HPB-LB Parenteral Grade (Roquette Frères, #346115; accessible at roquette.com/-/media/roquette-sharepoint-libraries/sdol_product-specification-sheet/roquette_quality_specification -sheet_kleptose-hpb-lb-parenteral-grade_50_346115_en.pdf as of Aug. 26, 2020), Cavitron® W7 HP5 Pharma cyclodextrin (Ashland; accessible at ashland.com/file_source/Ashland/Product/Documents/Pharmaceutical/PC_11734_Cavitron_Cav asol.pdf as of Aug. 26, 2020), Cavitron® W7 HP7 Pharma cyclodextrin (Ashland; accessible at ashland.com/file_source/Ashland/Product/Documents/Pharmaceutical/PC_11734_Cavitron_Cav asol.pdf as of Aug. 26, 2020), Trappsol® Cyclo™ (Cyclo Therapeutics, Inc.; accessible at cyclotherapeutics.com/cyclodextrins/trappsol-cyclo as of Aug. 26, 2020), and VTS-270/adrabetadex.

In certain embodiments, a cyclodextrin provided or used in a (e.g., pharmaceutical) composition or method or other application herein is a mixture of cyclodextrins; for example, in some embodiments, a 2-hydroxypropyl-beta-cyclodextrin provided herein comprises a mixture of 2-hydroxypropyl-beta-cyclodextrins. In some embodiments, a cyclodextrin molecule provided herein is optionally substituted with one or more chemical group, each chemical group independently being a hydroxypropyl group, a hydroxyethyl group, a methyl group, an ethyl group, a carboxymethyl group, a heptakis(2,6-di-O-methyl) group, a sulfoethyl group, a sulfopropyl group, and/or a sulfobutyl ethyl group, or its oligomer thereof. In some preferred embodiments, the cyclodextrin is a hydroxypropyl-beta-cyclodextrin, such as wherein one or more hydroxyl of the cyclodextrin is substituted with hydroxypropyl (e.g., 2-hydroxypropyl group). For example, one or more hydroxyl positions are substituted by one or more hydroxypropyl groups by substituting the H of the hydroxyl (OH) with a —CH₂CH₂(OH)CH₃ group, such as illustrated in Formula I below. In some embodiments, the 2-hydroxypropyl-beta -cyclodextrin comprises a plurality of cyclodextrins with various different Degree of Substitution (DS) values and/or having a Molar Substitution (MS) value.

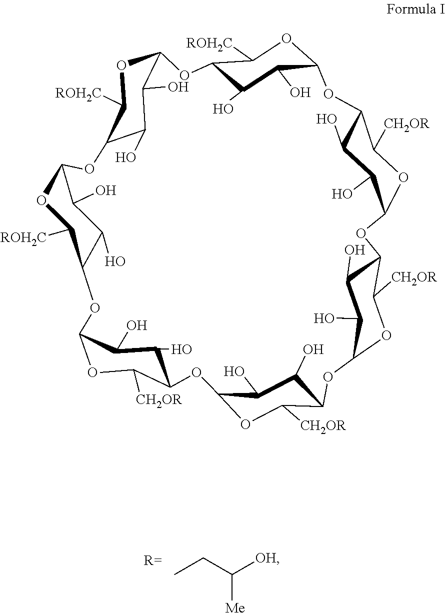

wherein each R is independently H or as noted above, and wherein at least one R is not H.

In some embodiments, the plurality of beta-cyclodextrin molecules in a beta-cyclodextrin (mixture of beta-cyclodextrin molecules) is characterized by an average molar substitution. The “molar substitution,” or “MS,” is the average number of substituents per glucose unit in the beta-cyclodextrin molecules. In some embodiments, MS is determined according to the procedures set forth in the USP monograph on Hydroxypropyl Betadex (USP NF 2015) (“USP Hydroxypropyl Betadex monograph”), incorporated herein by reference in its entirety. In some embodiments, the (e.g., pharmaceutical) compositions provided herein contain a plurality of beta-cyclodextrin molecules having an average MS of at least about 0.3. In some embodiments, the (e.g., pharmaceutical) compositions provided herein contain a plurality of beta-cyclodextrin molecules having an average MS of about 0.3 to 1.0.

In some embodiments, the plurality of beta-cyclodextrin molecules is characterized by average degree of substitution. The term “degree of substitution,” or “DS,” refers to the total number of substituents substituted directly or indirectly on a beta-cyclodextrin molecule. In some embodiments, the beta-cyclodextrin molecule may have one, or multiple, glucose units that are substituted by a substituent at a hydroxyl position. Thus, average DS refers to the total number of substituents in a population of beta-cyclodextrins divided by the number of beta-cyclodextrin molecules. In some embodiments, the average DS of the molecule is measured using Electron Spray Ionization-Mass Spectrometry (ESI-MS) analysis (e.g., HPLC-ESI-MS, etc.). In some embodiments, the average DS of the molecule is determined by peak heights of an electrospray MS spectrum. In some embodiments, the average DS of the molecule is determined by multiplying the MS by 7. In some embodiments, the (e.g., pharmaceutical) compositions provided herein contain a plurality of beta-cyclodextrin molecules having an average DS of about 2.0 to 7.0.

In some embodiments, any atom of the cyclodextrins described herein (e.g., 2-hydroxypropyl -beta-cyclodextrin) may be substituted with any suitable isotope. In a particular embodiment, any one or more hydrogen atoms of the cyclodextrins described herein (e.g., 2-hydroxypropyl -beta-cyclodextrin) may be substituted or replaced with deuterium atoms. Such cyclodextrins are expected to have similar or improved properties as compared to the original cyclodextrin that does not contain deuterium. Deuterium is a safe, stable, non-radioactive isotope of hydrogen. Compared to hydrogen, deuterium forms stronger bonds with carbon. In some instances, the increased bond strength imparted by deuterium can positively impact properties of the cyclodextrins, creating the potential for improved drug efficacy, safety, and/or tolerability. In addition, deuteration may cause decreased metabolic clearance in vivo, thereby increasing the half-life and circulation of the compound. At the same time, because the size and shape of deuterium are essentially identical to those of hydrogen, replacement of hydrogen by deuterium would not be expected to affect the biochemical potency and selectivity of the compound as compared to the original chemical entity that contains only hydrogen.

In various aspects, a therapeutically effective amount of 2-hydroxypropyl-beta-cyclodextrin is administered to the subject. In some embodiments, administration of a therapeutically effective amount of 2-hydroxypropyl-beta-cyclodextrin increases a circulating and/or systemic level of one or more derivative of cholesterol as compared to a baseline. In some embodiments, the one or more derivative of cholesterol is a by-product of cholesterol biosynthesis. In some embodiments, the one or more derivative of cholesterol comprises a hydrogenated product, products with differently hydrogenated 1H-cyclopenta[a]phenanthren-3-ol products, or products formed with a hydroxyl, epoxyl, or keto group. In some cases, the one or more derivative of cholesterol is an oxysterol or a sterol.

A therapeutically effective amount may be an amount of 2-hydroxypropyl-beta-cyclodextrin effective to increase a circulating and/or a systemic amount of one or more sterols and/or oxysterols in the subject as compared to a baseline. A circulating and/or systemic amount of a sterol and/or oxysterol may be an amount present in a biological sample of the subject (e.g., blood (e.g., whole blood), plasma, serum, and the like). In some cases, the level of a circulating and/or systemic sterol and/or oxysterol may be increased by at least about 10% as compared to a baseline (e.g., at 24 hours after treatment), (e.g., at least about 10%, at least about 15%, at least about 20%, at least about 25%, at least about 30%, at least about 35%, at least about 40%, at least about 45%, at least about 50%, or greater). In some cases, the level of a circulating and/or systemic sterol and/or oxysterol may be increased to at least about 40 ng/mL (e.g., at least about 40 ng/mL, at least about 50 ng/mL, at least about 60 ng/mL, at least about 70 ng/mL, at least about 80 ng/mL, at least about 90 ng/mL, at least about 100 ng/mL, or greater). In another aspect, the level of a circulating and/or systemic sterol and/or oxysterol may be increased to at least about 40 ng per mg of total circulating and/or systemic cholesterol (e.g., at least about 40 ng per mg, at least about 50 ng per mg, at least about 60 ng per mg, at least about 70 ng per mg, at least about 80 ng/mg, at least about 90 ng per mg, or at least about 100 ng per mg of total circulating and/or systemic cholesterol). Generally, the circulating and/or systemic levels of sterols and oxysterols (e.g., after treatment with 2-hydroxypropyl-beta-cyclodextrin) are compared to a baseline level (e.g., a circulating and/or systemic level of the sterol and/or oxysterol in the subject prior to treatment with the 2-hydroxypropyl-beta-cyclodextrin). Non-limiting examples of sterols and oxysterols that may demonstrate increased levels (e.g., in the whole blood, plasma, and/or serum) by administration of 2-hydroxypropyl-beta-cyclodextrin include: 27-hydroxycholesterol, 24-hydroxycholesterol, and 25-hydroxycholesterol. In various aspects, the therapeutically effective amount of 2-hydroxypropyl-beta-cyclodextrin may be an amount sufficient to maintain or sustain the systemic and/or circulating level of the sterol and/or oxysterol for at least 24 hours after treatment (e.g., at least 36 hours, at least 48 hours, at least 72 hours, at least 96 hours).

In a particular aspect, a therapeutically effective amount of 2-hydroxypropyl-beta-cyclodextrin is an amount effective to increase a circulating and/or systemic amount of 27-hydroxycholesterol as compared to a baseline. In some cases, the level of circulating and/or systemic 27-hydroxycholesterol may be increased by at least about 10% as compared to a baseline (e.g., at 24 hours after treatment), for example, increased by at least about 15%, at least about 20%, at least about 25%, at least about 30%, at least about 35%, at least about 40%, at least about 45%, at least about 50%, or greater. In some cases, the level of circulating and/or systemic 27-hydroxycholesterol may be increased to at least about 40 ng/mL, for example, to at least about 50 ng/mL, to at least about 60 ng/mL, to at least about 70 ng/mL, to at least about 80 ng/mL, to at least about 90 ng/mL, to at least about 100 ng/mL, or greater. In a particular embodiment, the level of circulating and/or systemic 27-hydroxycholesterol may be increased to at least about 100 ng/mL. In another aspect, the level of circulating and/or systemic 27-hydroxycholesterol may be increased to at least about 40 ng per mg of total circulating and/or systemic cholesterol, for example, to at least about 50 ng per mg, to at least about 60 ng per mg, to at least about 70 ng per mg, to at least about 80 ng per mg, to at least about 90 ng per mg, or to at least about 100 ng per mg of total circulating and/or systemic cholesterol. In a particular aspect, the level of circulating and/or systemic 27-hydroxycholesterol may be increased to at least about 90 ng per mg of total circulating and/or systemic cholesterol.

Additionally or alternatively, a therapeutically effective amount of 2-hydroxypropyl-beta -cyclodextrin may be an amount effective to increase a circulating and/or systemic amount of 24-hydroxycholesterol as compared to a baseline. In some cases, the level of circulating and/or systemic 24-hydroxycholesterol may be increased by at least about 10% as compared to a baseline (e.g., at 24 hours after treatment), for example, increased at least about 15%, at least about 20%, at least about 25%, at least about 30%, at least about 35%, at least about 40%, at least about 45%, at least about 50%, or greater. In some cases, the level of circulating and/or systemic 24-hydroxycholesterol may be increased to at least about 40 ng/mL, for example, to at least about 50 ng/mL, to at least about 60 ng/mL, to at least about 70 ng/mL, to at least about 80 ng/mL, to at least about 90 ng/mL, to at least about 100 ng/mL, or greater. In a particular embodiment, the level of circulating and/or systemic 24-hydroxycholesterol may be increased to at least about 50 ng/mL. In another aspect, the level of circulating and/or systemic 24-hydroxycholesterol may be increased to at least about 40 ng per mg of total circulating and/or systemic cholesterol, for example, to at least about 50 ng per mg, to at least about 60 ng per mg, to at least about 70 ng per mg, to at least about 80 ng/mg, to at least about 90 ng per mg, or to at least about 100 ng per mg of total circulating and/or systemic cholesterol. In a particular aspect, the level of circulating and/or systemic 24-hydroxycholesterol may be increased to at least about 40 ng per mg of total circulating and/or systemic cholesterol.

A therapeutically effective amount may be an amount of 2-hydroxypropyl-beta-cyclodextrin effective to increase plasma cholesterol crystal dissolution capacity (CCDC) after the administering (e.g., 1 hour after the administering) as compared to prior to the administering. In some cases, the therapeutically effective amount is an amount of 2-hydroxypropyl-beta-cyclodextrin effective to increase plasma CCDC by at least about 10% (e.g., at 1 hour) after the administering as compared to prior to the administering, such as by at least about 15%, at least about 20%, at least about 25%, at least about 30%, at least about 35%, at least about 40%, at least about 45%, at least about 50%, or greater.

A therapeutically effective amount may be an amount of 2-hydroxypropyl-beta-cyclodextrin effective to increase mRNA levels of one or more LXR transcription factor-regulated genes (e.g., ABCA1, ABCG1) after the administering (e.g., 24 hours after the administering) as compared to prior to the administering. In some cases, the therapeutically effective amount is an amount of 2-hydroxypropyl-beta-cyclodextrin effective to increase mRNA levels of ABCA1 and/or ABCG1 by at least about 10% (e.g., at 24 hours) after the administering as compared to prior to the administering, such as by at least about 15%, at least about 20%, at least about 25%, at least about 30%, at least about 35%, at least about 40%, at least about 45%, at least about 50%, or greater.

In some embodiments, the therapeutically effective amount of 2-hydroxypropyl-beta-cyclodextrin is an amount suitable to achieve the therapeutic effect described herein. In some embodiments, the therapeutically effective amount is at least about 50 mg/kg, at least about 100 mg/kg, at least about 200 mg/kg, at least about 300 mg/kg, at least about 400 mg/kg, at least about 500 mg/kg, at least about 600 mg/kg, at least about 700 mg/kg, at least about 800 mg/kg, at least about 900 mg/kg, at least about 1000 mg/kg, at least about 1100 mg/kg, at least about 1200 mg/kg, at least about 1300 mg/kg, at least about 1400 mg/kg, at least about 1500 mg/kg, at least about 1600 mg/kg, at least about 1700 mg/kg, at least about 1800 mg/kg, at least about 1900 mg/kg, or at least about 2000 mg/kg. In some embodiments, the therapeutically effective amount of 2-hydroxypropyl-beta-cyclodextrin is at least about 100 mg/kg. In some embodiments, the therapeutically effective amount of 2-hydroxypropyl-beta-cyclodextrin is at least about 250 mg/kg. In some embodiments, the therapeutically effective amount of 2-hydroxypropyl -beta-cyclodextrin is at least about 500 mg/kg. In some embodiments, the therapeutically effective amount of 2-hydroxypropyl-beta-cyclodextrin is at least about 1000 mg/kg. In some embodiments, the therapeutically effective amount of 2-hydroxypropyl-beta-cyclodextrin is at least about 1500 mg/kg.

In some embodiments, the therapeutically effective amount of 2-hydroxypropyl-beta-cyclodextrin is an amount suitable to achieve the therapeutic effect described herein. In some embodiments, the therapeutically effective amount is from about 50 mg/kg to about 2000 mg/kg (e.g., from about 50 mg/kg to about 1000 mg/kg, from about 500 mg/kg to about 1000 mg/kg, from about 500 mg/kg to about 1500 mg/kg, from about 800 mg/kg to about 1500 mg/kg, from about 800 mg/kg to about 1200 mg/kg, from about 1000 mg/kg to about 1500 mg/kg, from about 1000 mg/kg to about 2000 mg/kg. In some embodiments, the therapeutically effective amount of 2-hydroxypropyl-beta-cyclodextrin is from about 500 mg/kg to about 1500 mg/kg. In some embodiments, the therapeutically effective amount of 2-hydroxypropyl-beta-cyclodextrin is from about 800 mg/kg to about 1200 mg/kg.

In some embodiments, the therapeutically effective amount of 2-hydroxypropyl-beta-cyclodextrin is an amount suitable for achieving the therapeutic effect described herein. In some embodiments, the therapeutically effective amount is at least about 4 g (e.g., at least about 10 g, at least about 25 g, at least about 50 g, at least about 75 g, at least about 100 g, at least about 125 g, at least about 150 g, at least about 175 g, at least about 200 g, at least about 250 g). In some embodiments, the therapeutically effective amount of 2-hydroxypropyl-beta-cyclodextrin may be from about 4 g to about 250 g (e.g., from about 4 g to about 200 g, from about 4 g to about 150 g, from about 4 g to about 100 g, from about 4 g to about 50 g, from about 50 g to about 250 g, from about 50 g to about 200 g, from about 50 g to about 150 g, from about 50 g to about 100 g, from about 100 g to about 250 g, from about 100 g to about 200 g). The total amount of 2-hydroxpropyl -beta-cyclodextrin administered (e.g., in a single dose administration, e.g., in a therapeutically effective amount) may depend on a number of factors, including, without limitation, the subject's age, gender, weight, and the like.

In some embodiments, the therapeutically effective amount of 2-hydroxypropyl-beta-cyclodextrin is an amount sufficient to achieve a whole blood, serum, and/or plasma concentration of 2-hydroxypropyl-beta-cyclodextrin suitable for achieving the therapeutic effect described herein. In some embodiments, the whole blood, serum, and/or plasma concentration is at least about 0.1 mM (e.g., at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1.0 mM, at least about 1.5 mM, at least about 2.0 mM, at least about 2.5 mM, or at least about 3 mM). The therapeutically effective amount of 2-hydroxypropyl -beta-cyclodextrin may be an amount sufficient to achieve a whole blood, serum, and/or plasma concentration of 2-hydroxypropyl-beta-cyclodextrin of about 0.6 mM to about 3 mM (e.g., about 0.6 mM to about 2 mM, about 0.6 mM to about 1 mM, about 1 mM to about 3 mM, about 1 mM to about 2 mM, about 2 mM to about 3 mM).

The methods disclosed herein may further comprise administering, at a first time point, a therapeutically effective first amount of 2-hydroxypropyl-beta-cyclodextrin to a subject, and administering, at a second time point, a therapeutically effective second amount of 2-hydroxypropyl -beta-cyclodextrin to the subject. The second time point can be at least 1 day, 2 days, 3 days, 4 days, 5 days, or 6 days after the first time point. The second time point can be at least 1 week after the first time point (e.g., 2 weeks, 3 weeks, 4 weeks, 5 weeks, 6 weeks, 7 weeks, 8 weeks, 9 weeks, or 10 weeks after the first time point). In some cases, the second time point is one or more week after the first time point, two weeks after the first time point, or one or more month after the first time point. In some embodiments, the administering may be by intravenous administration.

In some cases, the second time point may be determined based on one or more indicators that an additional dose of drug would be beneficial to the subject. For example, the second time point may be administered after the therapeutic benefit of the first dose has diminished or has started to diminish. The second time point can be determined based on, e.g., levels of circulating and/or systemic 27-hydroxycholesterol, levels of circulating and/or systemic 24S-hydroxycholesterol levels, transcription levels of ATP-binding cassette subfamily A member 1 (ABCA1), transcription levels of ATP-binding cassette subfamily G member 1 (ABCG1), epigenetic (chromatin) signature of peripheral blood mononuclear cells (PBMCs), triglyceride levels, total cholesterol levels, very low-density lipoprotein (vLDL) level, low density lipoprotein (LDL) level, high density lipoprotein (HDL) level, serum or plasma cholesterol crystal dissolution, levels of pro-inflammatory mediators (e.g., interleukin-1b (IL-1b), interleukin-6 (IL-6), interleukin-18 (IL-18)).

In various aspects, the subject can be a human. In some cases, the subject may be of any age that is at risk of or more prone to developing atherosclerosis and/or atherosclerotic cardiovascular disease (e.g., CAD). The subject may be at least 30 years old (e.g., at least 40, at least 50 at least 60, at least 70, at least 80, at least 90 years old). The subject may be less than 30 years old (e.g., less than 20 years old, less than 15 years old, less than 10 years old, or less than 5 years old). The subject can be diagnosed with atherosclerosis and/or atherosclerotic cardiovascular disease (e.g., CAD). The subject can be diagnosed with acute coronary syndrome (ACS) or chronic coronary syndrome (CCS) (e.g., as defined by the European Society of Cardiology). Atherosclerosis and/or atherosclerotic cardiovascular disease can be diagnosed via e.g., angiogram, cholesterol test, a computed tomography (CT) scan, Duplex scanning, an echocardiogram, an electrocardiogram (ECG or EKG), exercise stress test, an intravascular ultrasound, a magnetic resonance imaging (MRI) scan, a positron emission tomography (PET) scan, an optical coherence tomography (OCT) scan, a pharmacologic stress test, symptoms/medical history (e.g., patient-reported symptoms), fat attenuation index (FAI), or a combination thereof. The subject can have a symptom associated with atherosclerosis and/or atherosclerotic cardiovascular disease. The symptom associated with atherosclerosis and/or atherosclerotic cardiovascular disease can be chest pain (e.g., angina), shortness of breath, fatigue, confusion, muscle weakness, or a combination thereof. The subject can be at risk of developing atherosclerosis and/or atherosclerotic cardiovascular disease. A subject at risk of developing atherosclerosis and/or atherosclerotic cardiovascular disease can have at least one atherosclerosis risk factor. In some embodiments, atherosclerosis risk factors include, without limitation, being overweight or obese, high blood pressure, high cholesterol level, diabetes, lack of physical activity, one or more co-morbidities (e.g., smoking, renal disease, rheumatoid disease), and the use of chemotherapeutic agents. The subject can have at least a 10%, 20%, 30%, 40%, 50%, 60%, 70%, 80%, or 90% stenosis of an artery and/or a vein. The subject can have at least a 50% steno sis of an artery and/or a vein. The artery can be, e.g., a coronary artery, a cerebral artery, a peripheral artery, the aorta. The subject may have one or more atherosclerotic plaques having a high cholesterol and/or high lipid content (e.g., as measured by optical coherence tomography (OCT)). The subject may have one or more plaques having a low calcium score (e.g., as measured by a computed tomography (CT) scan). The subject may have stable or unstable CAD. The subject may have acute coronary syndrome (ACS) or chronic coronary syndrome (CCS) (e.g., as defined by the European Society of Cardiology). The subject may be treated (e.g., by the methods described herein) after (e.g., immediately after) having a myocardial infarction. The subject may have a thickening of the arterial wall (tunica media). The subject may be treated (e.g., by the methods described herein) after undergoing chemotherapy (e.g., the subject may have an increased risk of or may have developed atherosclerosis due to the use of chemotherapeutic agents).

The methods disclosed herein can be used to treat and/or prevent atherosclerotic cardiovascular disease. For example, the methods disclosed herein can be used to treat and/or prevent CAD, PAD, PVD, stroke, chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, and/or post-surgical or iatrogenic arterial disease. The methods disclosed herein can be used to treat and/or prevent atherosclerosis.

In some embodiments, the methods described herein causes a reduction in the size of atherosclerotic plaques (e.g., cholesterol-rich plaques and/or lipid-rich plaques) in the subject. The plaques may have a high cholesterol and/or high lipid content. The cholesterol and/or lipid content may be measured by, e.g., optical coherence tomography (OCT). The plaques may have a low calcium content. The plaques may have a low calcium score on a computed tomography (CT) scan. In some cases, the size of an atherosclerotic plaque may be reduced relative to the size of the atherosclerotic plaque prior to the treating. In some embodiments, the size of an atherosclerotic plaque may be reduced by at least about 0.5%. In some embodiments, the size of an atherosclerotic plaque may be reduced by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or greater.

In some embodiments, the methods described herein cause a decrease in and/or prevent the progression and/or development of atherosclerosis in the subject. In some embodiments, the methods described herein prevent the progression of atherosclerosis. For example, the methods described herein prevent an increase in the size of atherosclerotic plaques, prevent an increase in plaque volume, prevent an increase in the amount of the coronary arterial surface covered by plaques, prevent thickening of the atherosclerotic plaques, prevent an increase in stenosis of an artery or vein, prevent or reduce thickening of an arterial wall, and/or prevent or reduce vascular calcification. In some cases, the methods described herein mediate the regression of already-developed atherosclerotic plaques in the subject. For example, the methods described herein reduce the size and/or number of atherosclerotic plaques already-developed in the subject, and/or reduce the size of the necrotic core of an atherosclerotic plaque.

The treating may result in an increase in flow-mediated vasodilation (FMD) of the brachial artery. The increase in flow-mediated vasodilation (FMD) can occur at least 7 days after the treating. Flow-mediated vasodilation (FMD) can be measured by, e.g., ischemia-induced endothelial-dependent vasodilation. In some embodiments, flow-mediated vasodilation (FMD) is determined using high resolution ultrasonography. In some cases, the treating may result in an increase in sterol and/or oxysterol levels (e.g., 27-hydroxycholesterol, 24-hydroxycholesterol) in whole blood, serum, plasma, or any combination thereof. In some cases, the treating may result in the dissolution of cholesterol crystals e.g., present in atherosclerotic plaques. Cholesterol crystal dissolution can be measured by, e.g., a cholesterol crystal dissolution capacity assay. In some cases, the treating may result in an increase in gene expression of a liver X receptor (LXR)-regulated gene. The increase in the LXR-regulated gene can be in, e.g., peripheral blood mononuclear cells (PBMCs) of the subject. The LXR-regulated gene can be, e.g., ATP-binding cassette subfamily A member 1 (ABCA1), ATP-binding cassette subfamily G member 1 (ABCG1), fatty acid synthase (FAS), apolipoprotein E (APOE), or a combination thereof. In some cases, the treating may result in an increase in the phagocytic activity of PBMCs. In some cases, the treating may result in an increase or a decrease in a level of a lipid in a biological sample from the subject. The lipid can be a triglyceride, LDL-cholesterol, HDL-cholesterol, or apolipoprotein A1 (ApoA1). The treating can result in a decrease in a level of triglycerides in the biological sample. The treating can result in a decrease of LDL-cholesterol in the biological sample. The treating can result in an increase in a level of HDL-cholesterol in the biological sample. The treating can result in an increase in a level of ApoA1 in the biological sample. The biological sample can be blood (e.g., whole blood, serum, plasma). In some cases, the treating may result in a decrease in serum markers of inflammation and myocardial damage and/or an increase in serum markers of anti-inflammation. The serum marker can be interleukin (IL)-1 beta (IL-1beta), interleukin-1 receptor antagonist (IL-1ra), interleukin-1 alpha (IL-1a), interleukin-6 (IL-6), highly sensitive C-reactive protein (hsCRP), Troponin, creatine kinase (CK), creatine kinase-MB (CK-MB), N-terminal pro-B-type natriuretic peptide (NT-pro-BNP). In some cases, the treating may result in a decrease in complement activation. In some cases, the treating may result in a decrease in a risk of mortality and/or all-cause mortality (ACM) of the subject. In some embodiments, the risk of mortality of the subject is decreased by 25%, 50%, 75%, or 90%. In some embodiments, the risk of mortality of the subject is decreased for at least 1 year, 2 years, or 3 years after the administering. In some cases, the treating may result in a decrease in a risk of myocardial infarction in the subject. In some embodiments, the risk of myocardial infarction in the subject is decreased by 25%, 50%, 75%, or 90%. In some embodiments, the risk of myocardial infarction in the subject is decreased for at least 1 year, 2 years, or 3 years after the administering. In some cases, the treating may result in a decrease in a risk of major or minor stroke in the subject. In some embodiments, the risk of major or minor stroke in the subject is decreased by 25%, 50%, 75%, or 90%. In some embodiments, the risk of major or minor stroke in the subject is decreased for at least 1 year, 2 years, or 3 years after the administering. In some cases, the treating may result in a decrease in a blood pressure of the subject. In some embodiments, the blood pressure of the subject is decreased by at least 5%, 10%, 15%, 20%, 25%, or 30%. The decrease in blood pressure can comprise a decrease in systolic blood pressure, diastolic blood pressure, or a combination thereof. In some cases, the treating may result in a decrease in a risk of a major adverse cardiovascular event (MACE) in the subject. In some embodiments, a major adverse cardiovascular event comprises heart failure, re-infarction, recurrent angina pain, re-hospitalization for cardiovascular-related illness, repeat percutaneous coronary intervention (PCI), coronary artery bypass grafting, coronary revascularization, stroke, all-cause mortality (ACM), or a combination thereof. In some embodiments, the risk of MACE in the subject is decreased by 25%, 50%, 75%, or 90%. In some embodiments, the risk of MACE in the subject is decreased for at least 1 year, 2 years, or 3 years after the administering. In some cases, the treating may result in improved erectile dysfunction, in correlation to CAD severity.

In some cases, the treating may result in a level of a liver enzyme less than 2.5 times up to a normal level of the liver enzyme. The liver enzyme can be alanine aminotransferase (ALT), aspartate aminotransferase (AST), or the combination thereof. The liver enzyme can be alanine aminotransferase (ALT), aspartate aminotransferase (AST), alkaline phosphatase (ALP), 5′ nucleotidase, gamma-glutamyl transpeptidase (GGT), or a combination thereof. In some embodiments, the normal level of the liver enzyme is a level of the liver enzyme in the absence of damage to the liver. In some cases, the treating may result in serum creatinine levels less than 0.3 mg/dl. The treating may result in serum creatinine levels less than 1.3, 1.0, 0.75, 0.5, or 0.3 mg/dl. In some cases, the treating may result in no substantial loss of sensorineural hearing.

Pharmaceutical Compositions

Disclosed herein, in certain embodiments, are pharmaceutical compositions comprising an amount of 2-hydroxypropyl-beta-cyclodextrin effective to treat atherosclerotic cardiovascular disease and/or atherosclerosis in a human; and an excipient. The excipient can be a pharmaceutically acceptable excipient.

The pharmaceutical composition may comprise an amount of 2-hydroxypropyl-beta-cyclodextrin effective to increase a circulating and/or systemic level of one or more oxysterol in a subject by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to increase plasma cholesterol crystal dissolution capacity (CCDC) by at least about 10% (e.g., at 1 hour) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to increase mRNA levels of one or more LXR transcription factor-regulated genes (e.g., ABCA1 and/or ABCG1) by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The excipient may comprise a tonicity adjusting agent, a preservative, a solubilizing agent, a buffer, a solution (e.g., an IV solution), or any combination thereof. The tonicity adjusting agent can be dextrose, glycerol, sodium chloride, glycerin, mannitol, or a combination thereof. The preservative can be an antioxidant, an antimicrobial, a chelating agent, or a combination thereof. The antioxidant can be ascorbic acid, acetylcysteine, a sulfurous acid salt (e.g., bisulfite, metabisulfite), a monothioglycerol, or a combination thereof. The antimicrobial can be a phenol, meta-cresol, benzyl alcohol, paraben, benzalkonium chloride, chlorobutanol, thimerosal, phenylmercuric salts (e.g., acetate, borate, nitrate), or a combination thereof. The chelating agent can be calcium disodium ethylenediaminetetraacetic acid (EDTA), disodium EDTA, sodium EDTA, calcium versetamide sodium, calteridol, diethylenetriaminepenta acetic acid (DTPA), or a combination thereof. The solubilizing agent can be a surfactant or a co-solvent. The surfactant can be polyoxyethylene sorbitan monooleate (Tween 80), sorbitan monooleate polyoxyethylene sorbitan monolaurate (Tween 20), lecithin, polyoxyethylene-polyoxypropylene copolymers (Pluronics), or a combination thereof. The co-solvent can be propylene glycol, glycerin, ethanol, polyethylene glycol (PEG), sorbitol, dimethylacetamide, Cremophor EL, or a combination there. The polyethylene glycol can be PEG 300, PEG 400, PEG 600, PEG 3350, or PEG 4000. The buffer can comprise sodium acetate, acetic acid, glacial acetic acid, ammonium acetate, ammonium sulfate, ammonium hydroxide, arginine, aspartic acid, benzene sulfonic acid, benzoate sodium, benzoic acid, sodium bicarbonate, boric acid, sodium boric acid, sodium carbonate, citrate acid, sodium citrate, disodium citrate, trisodium citrate, diethanolamine, glucono delta lactone, glycine, glycine HCl, histidine, histidine HCl, hydrochloric acid, hydrobromic acid, lysine, maleic acid, meglumine, methanesulfonic acid, monoethanolamine, phosphate acid, monobasic potassium, dibasic potassium, monosodium phosphate, disodium phosphate, trisodium phosphate, sodium hydroxide, succinate sodium, sulfuric acid, tartarate sodium, tartaric acid, tromethamine (Tris), or a combination thereof.

The pharmaceutical composition can comprise at least about 4, at least about 10, at least about 50, at least about 100, at least about 150, at least about 200, or at least about 250 g of 2-hydroxypropyl -beta-cyclodextrin. In some embodiments, the pharmaceutical composition comprises at least about 4 g of 2-hydroxypropyl-beta-cyclodextrin. In some embodiments, the pharmaceutical composition comprises at least about 50 g of 2-hydroxypropyl-beta-cyclodextrin. In some embodiments, the pharmaceutical composition comprises at least about 100 g of 2-hydroxypropyl-beta-cyclodextrin. In some embodiments, the pharmaceutical composition comprises at least about 200 g of 2-hydroxypropyl-beta-cyclodextrin. In some embodiments, the pharmaceutical composition comprises from about 4 g to about 250 g of 2-hydroxypropyl -beta-cyclodextrin (e.g., from about 4 g to about 100 g, from about 4 g to about 50 g, from about 50 g to about 150 g, from about 50 g to about 250 g, from about 100 g to about 200 g, from about 100 g to about 250 g, from about 150 g to about 250 g.)

The pharmaceutical composition can be formulated for single dose administration. The pharmaceutical composition can be formulated for intravenous administration. The pharmaceutical composition can be formulated to be isotonic.

Kits

Further provided herein are kits. In some cases, the kits include one or more container (e.g., a vial, a flask, a jar, a tube, an ampoule, etc.) containing one or more pharmaceutical compositions provided herein (e.g., 2-hydroxypropyl-beta-cyclodextrin and a pharmaceutically acceptable excipient). In some cases, the kit comprises more than one container (e.g., two, three, four, five, six, seven, eight, nine, ten, or more containers). In some cases, at least one of the one or more container is an IV infusion bag. The one or more container may include a single dosage of the pharmaceutical composition, or multiple dosages (e.g., two, three, four, five, six, seven, eight, nine, ten, or more) of the pharmaceutical composition. In some cases, the one or more container contains a concentrated amount of the pharmaceutical composition which is subsequently diluted, prior to administration, to achieve an effective dosage. The dosage may be any amount as described herein, effective to treat one or more indications described herein. The kit may further comprise one or more additional components for IV infusion of the pharmaceutical composition. In some cases, the kit comprises an IV infusion bag. In some cases, the kit comprises one or more solutions (e.g., saline) for mixing and/or diluting the pharmaceutical composition. In some cases, the kit comprises one or more of a catheter, a tubing, a syringe, and a needle. The kit may further comprise instructions, e.g., for administering the pharmaceutical composition to a subject for the use of treating any indication described herein (e.g., for the treatment of atherosclerosis and/or atherosclerotic cardiovascular disease in a human individual and/or reducing or inhibiting the development of cholesterol rich plaque in a human individual). The kit may be provided in a box, a bag, or any other suitable container.

In some aspects, the kit may comprise one or more additional active pharmaceutical ingredient (e.g., therapeutic compounds, drugs, etc.). In some cases, the kit may comprise a single container containing a pharmaceutical composition of the disclosure (e.g., 2-hydroxypropyl -beta-cyclodextrin and a pharmaceutically acceptable excipient) and the one or more additional active pharmaceutical ingredient. In other cases, the kit may comprise a first container containing a pharmaceutical composition of the disclosure (e.g., 2-hydroxypropyl-beta -cyclodextrin and a pharmaceutically acceptable excipient) and a second container containing the one or more additional active pharmaceutical ingredient.

### EXAMPLES

### Example 1

### Phase 1b and 2a Clinical Trial for Treatment of Patients with Advanced Atherosclerosis with 2-hydroxypropyl-beta-cyclodextrin

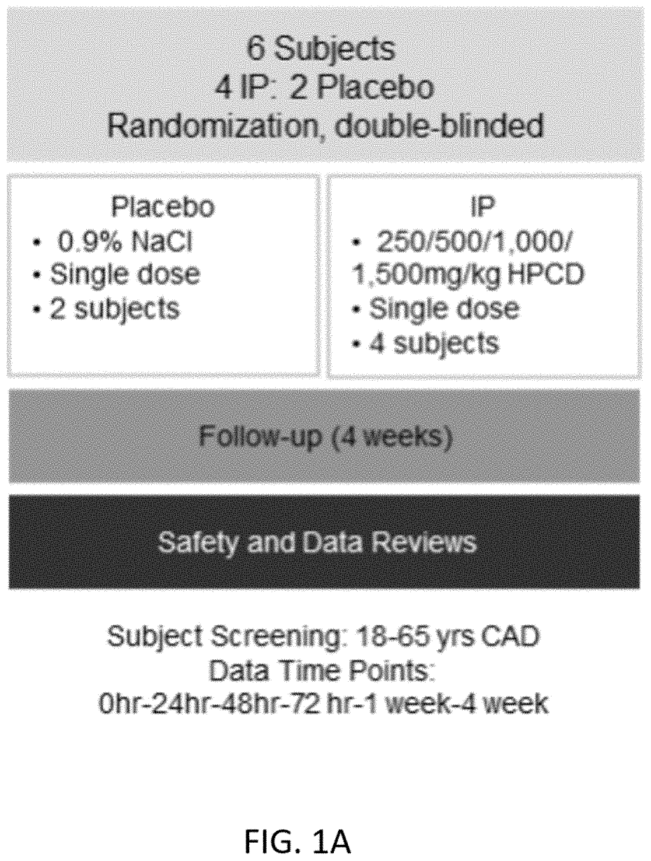

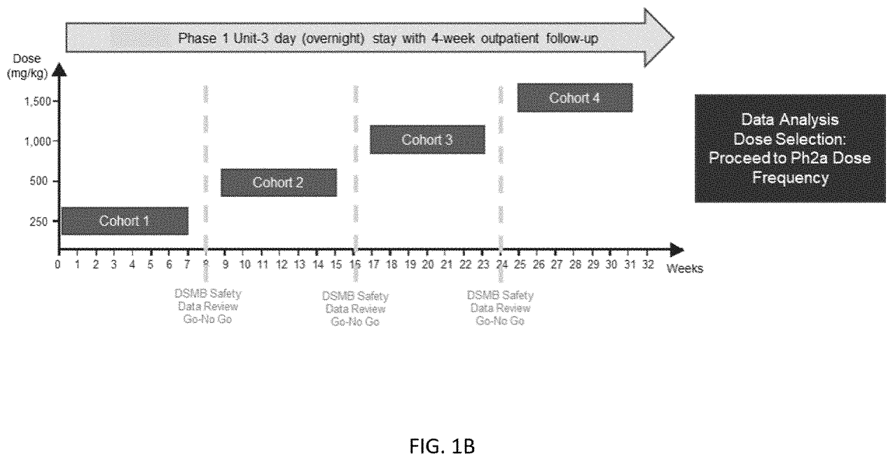

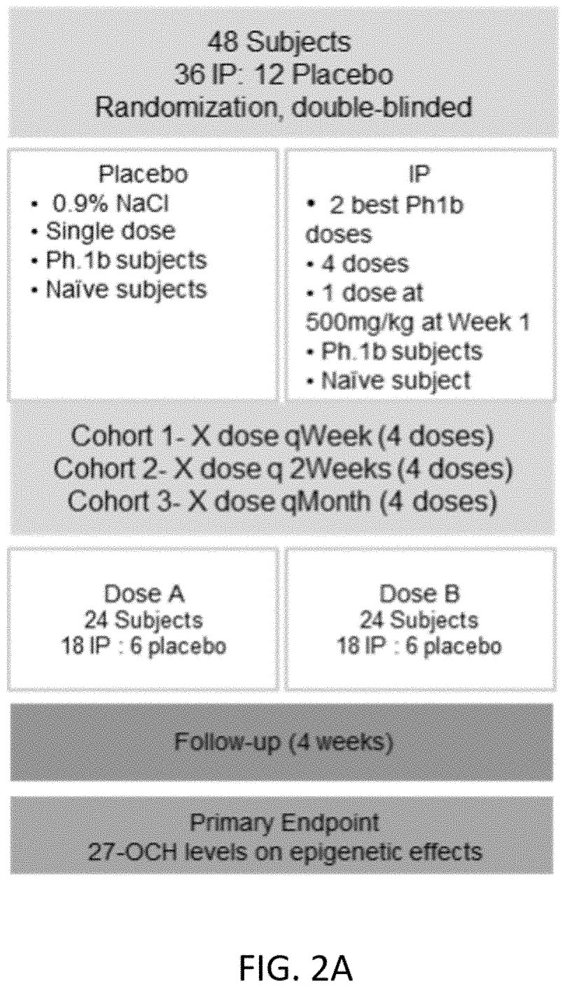

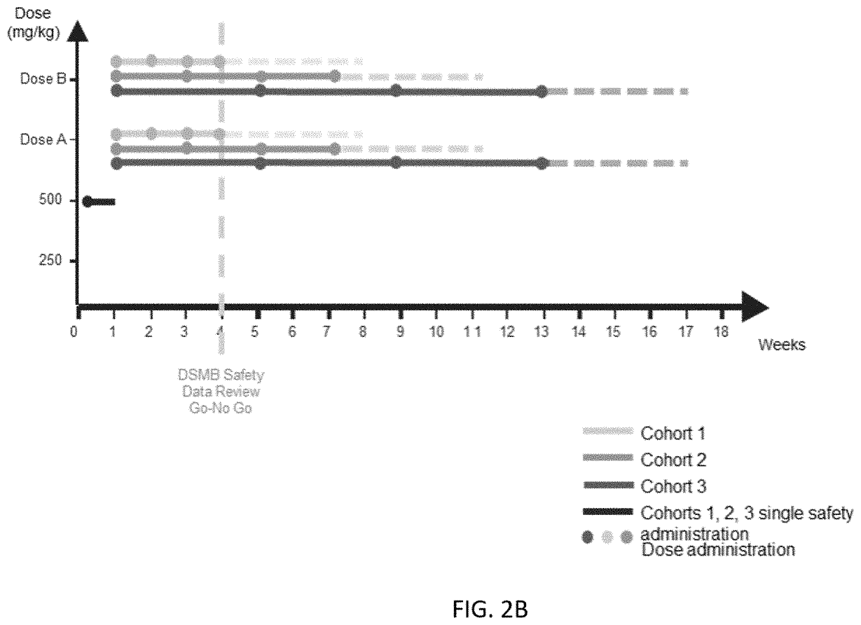

Males and females with stable coronary artery disease participate in a randomized, double blind, single-center, placebo controlled studies. Safety (Phase 1b) and efficacy (Phase 2a) of 2-hydroxypropyl-beta-cyclodextrin to induce 27-hydroxycholesterol levels and epigenetic changes, after single (Phase 1b) and repeated dosing (Phase 2a) is assessed. FIGS. 1A, 1B, 2A, and 2B further describe the administration of the placebo and investigational product (IP) for use in the Phase 1b and Phase 2a studies.

This randomized, double-blind, placebo-controlled trial is conducted at a single study center and has two parts.

Part A (Phase 1b) evaluates the safety and tolerability of 2-hydroxypropyl-beta-cyclodextrin, pharmacokinetics of 2-hydroxypropyl-beta-cyclodextrin, and explores the effects on biomarker endpoints in up to four sequential cohorts of CAD patients administered single-ascending IV doses of 2-hydroxypropyl-beta-cyclodextrin. After each cohort, safety and tolerability data is reviewed prior to further dose escalation in the next cohort. Dose escalation may continue up to the maximum well-tolerated dose, but does not exceed plasma 2-hydroxypropyl -beta-cyclodextrin exposures pre-specified in the study protocol.

Part B (Phase 2a) evaluates the safety and tolerability of 2-hydroxypropyl-beta-cyclodextrin, pharmacokinetics of 2-hydroxypropyl-beta-cyclodextrin, and effects on 27-hydroxycholesterol measured at 48 hours post-infusion as the primary biomarker endpoint in up to three sequential cohorts of CAD patients with escalating, multiple IV doses of 2-hydroxypropyl -beta-cyclodextrin, administered at intervals for up to four weeks. Doses and dose intervals for Part B are chosen based on preliminary safety and tolerability, and PK and biomarker (PD) results from Part A.

The dose levels in Part A are: Group A1: subjects are administered either 250 mg/kg 2-hydroxypropyl -beta-cyclodextrin or placebo; Group A2: subjects are administered either 500 mg/kg 2-hydroxypropyl-beta-cyclodextrin or placebo; Group A3: subjects are administered either 1,000 mg/kg 2-hydroxypropyl-beta-cyclodextrin or placebo; and Group A4: subjects are administered either 1,500 mg/kg 2-hydroxypropyl-beta-cyclodextrin or placebo.

In Part B, each Dose Group (Dose A; Dose B) enrolls a total of 24 subjects, including at least four of each gender. Each Dose Group is randomized to three Dose Frequency arms (qweek; q2weeks; qmonth). In each Dose Frequency arm, six subjects are randomized to 2-hydroxypropyl -beta-cyclodextrin and two subjects to placebo. Group 1: subjects are administered either 2-hydroxypropyl-beta-cyclodextrin or placebo, 1× week, up to four doses; Group 2: subjects are administered either 2-hydroxypropyl-beta-cyclodextrin or placebo, 1× every two weeks, up to four doses; and Group 3: subjects are administered either 2-hydroxypropyl -beta-cyclodextrin or placebo, 1× month, up to four doses.

Primary Objectives

The primary endpoint determines the safety and tolerability of 2-hydroxypropyl-beta-cyclodextrin, administered as single and multiple IV doses.

Secondary Objectives

The secondary endpoint evaluates the single- and multiple-dose pharmacokinetics of 2-hydroxypropyl -beta-cyclodextrin.

Safety Endpoints

Safety endpoints include physical examinations, vital signs (e.g., blood pressure, heart rate, respiratory rate, temperature, body weight), 12-lead ECG, pulse oximetry SpO₂, routine clinical laboratory tests (e.g., hematology, chemistry, urinalysis), liver function test (eGFR), audiometry, and adverse events.

Pharmacodynamics Endpoints

Pharmacodynamics endpoints include cholesterol metabolites (e.g., 24-, 25-, 27-hydroxycholesterol) in blood and urine samples; cholesterol crystal dissolution capacity (CCDC) assay (ex vivo) in whole blood samples; plasma lipids (e.g., HDL-, LDL-cholesterol, total cholesterol, triglycerides) and ApoA1 levels; plasma myeloperoxidase (MPO) and fibrinogen; plasma markers of inflammation (e.g., IL-1β, IL-1a, IL-6, hsCRP) and/or plasma levels of anti-inflammatory cytokines (e.g., IL-1ra) and C3/C4 complement activity; gene expression (including LXR target genes), epigenomic assays, and phagocytosis assays (ex vivo) in isolated PBMCs; and flow-mediated dilatation (FMD) of brachial artery (e.g., non-invasive, quantitative ultrasound imaging method).

The efficacy and biological effects of HPCD treatment on specific organ and cell systems is also assessed by determining secondary endpoints. Secondary endpoints include: change in flow-mediated vasodilation (FMD) of the brachial artery, as assessed by ischemia-induced endothelium-dependent vasodilation after 7 days, using high-resolution ultrasonography; sterols/oxysterols (e.g. 24S- and 25-hydroxycholesterol levels in serum/plasma, urine, and stool); epigenomic data, ATAC sequencing of whole blood (PBMC); cholesterol crystal dissolution capacity (CCDC); change in gene expression of LXR-regulated genes in PMBCs; PBMC's phagocytic activity; MPO, fibrin; microbiome; pK of HPCD in serum, urine, stool; lipid levels (triglycerides, LDL-/HDL-cholesterol, ApoA1) in serum, urine, and stool; serum markers of inflammation and myocardial damage (IL-1β, IL-1a, IL-6, hsCRP, troponin, CK, CK-MB, and NT-pro-BNP); serum levels of anti-inflammatory cytokines (e.g., IL-1ra); complement activation; all-cause and cardiovascular mortality; myocardial infarction; major and minor stroke; and blood pressure (24h registration).

### Example 2

### Plasma Obtained from a Subject Treated with 2-hydroxypropyl-beta-cyclodextrin Demonstrates Cholesterol Crystal Dissolution Capacity

A male human subject was treated with 2-hydroxypropyl-beta-cyclodextrin with single-ascending doses administered intravenously every 4 weeks according to Table 1 below.

TABLE 1
Dose schedule

| | Amount of 2-hydroxypropyl- | |
|---|---|---|
| Dose | beta-cyclodextrin | |
| D 0 (Week 0) | 500 | mg/kg |
| M 1 (Week 4) | 750 | mg/kg |
| M 2 (Week 8) | 1,000 | mg/kg |
| M 3 (Week 12) | 1,000 | mg/kg |

For each dose, whole blood was collected pre-dose, post-dose (line flush), 1 hour post-dose, and 24 hours post-dose. Plasma was separated from the whole blood and subjected to a cholesterol crystal dissolution capacity (CCDC) assay using techniques similar to those described in the literature. The CCDC assay measures the ability of a sample to dissolve cholesterol crystals.

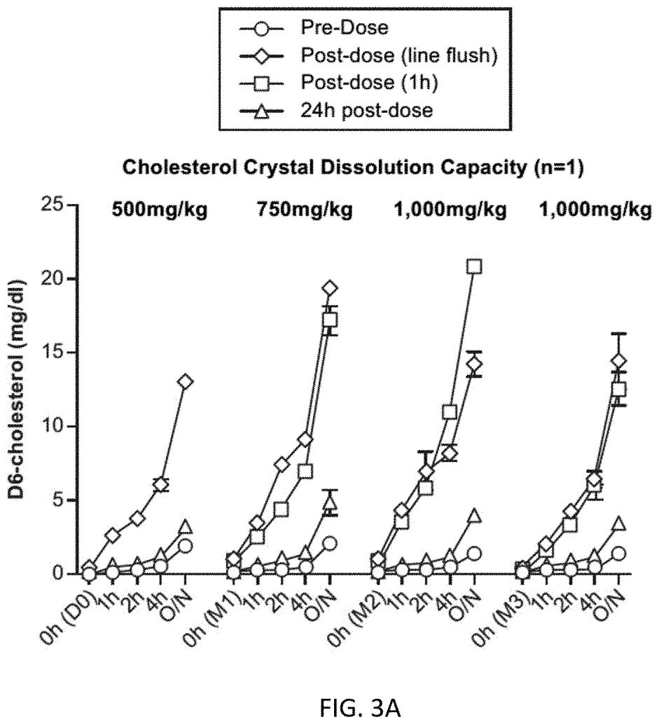

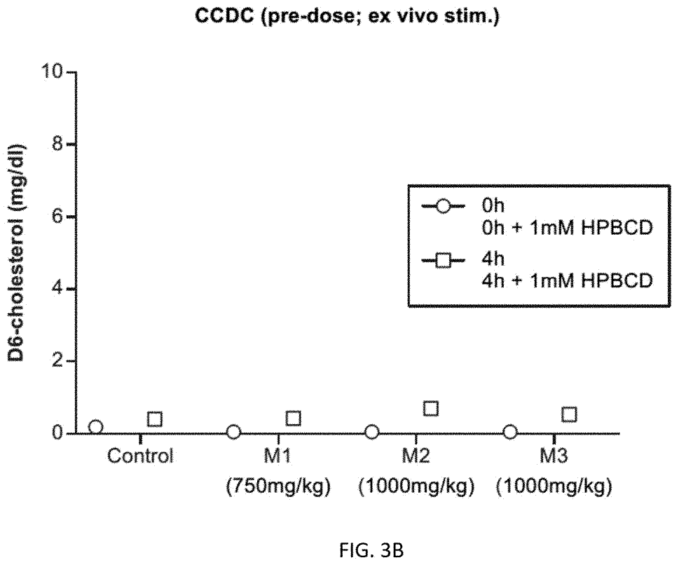

FIG. 3A depicts results of the CCDC assay. FIG. 3A demonstrates increased capability of blood plasma to dissolve cholesterol crystals after treatment with 2-hydroxypropyl-beta-cyclodextrin. This suggests that 2-hydroxpropyl-beta-cyclodextrin treatment increases plasma factors which bind and solubilize cholesterol crystals for cross-tissue transport accelerating reverse cholesterol transport (RCT). 2-hydroxypropyl-beta-cyclodextrin is able to restore and improve the plasma dissolution of free cholesterol, acceleration of reverse cholesterol transport has been shown to reduce atherosclerotic plaques, and the data further demonstrates that treatment with 2-hydroxypropyl-beta-cyclodextrin may be a suitable treatment for atherosclerosis and/or atherosclerotic cardiovascular disease.

Blood plasma pre-dose was also incubated ex vivo with 2-hydroxypropyl-beta-cyclodextrin and the ability of the plasma to dissolve cholesterol crystals was measured. FIG. 3B demonstrates that plasma treated ex vivo with 2-hydroxypropyl-beta-cyclodextrin demonstrates increased capacity to dissolve cholesterol crystals.

Taken together, the data presented herein demonstrates that treatment with 2-hydroxypropyl -beta-cyclodextrin increases plasma cholesterol crystal dissolution capacity which may lead to dissolution of and/or clearance of atherosclerotic plaques. The data further demonstrates that treatment with 2-hydroxypropyl-beta-cyclodextrin may be a suitable treatment for atherosclerosis and/or atherosclerotic cardiovascular disease, as described herein.

### Example 3

### Treatment with 2-hydroxypropyl-beta-cyclodextrin Increases Sterol and Oxysterol Concentrations in a Human Subject

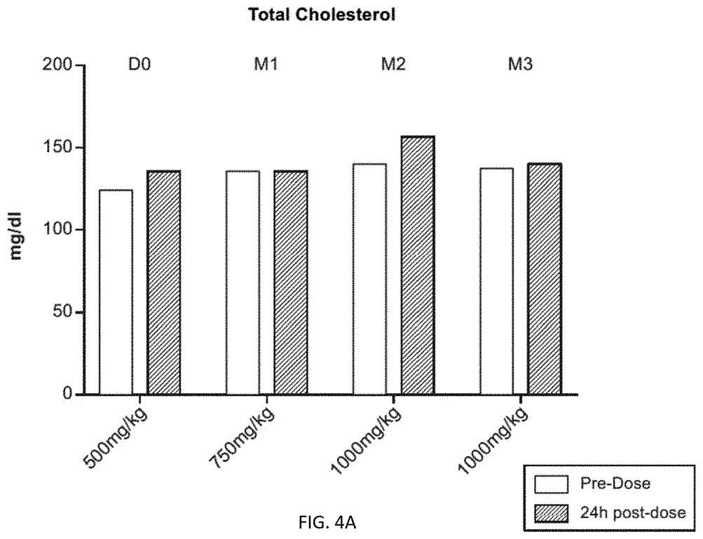

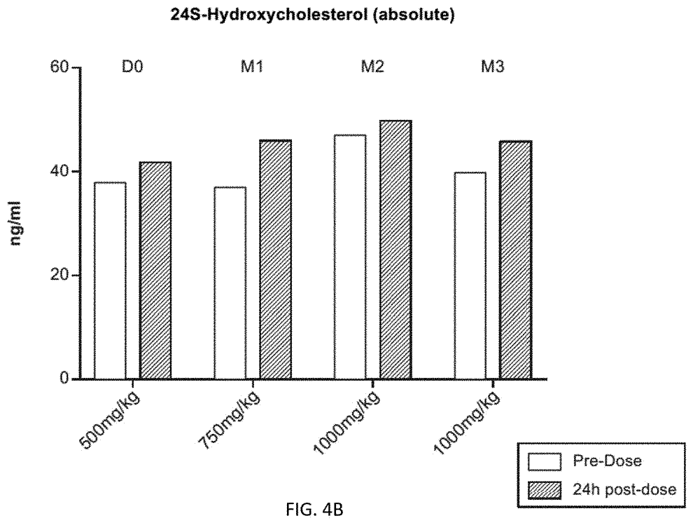

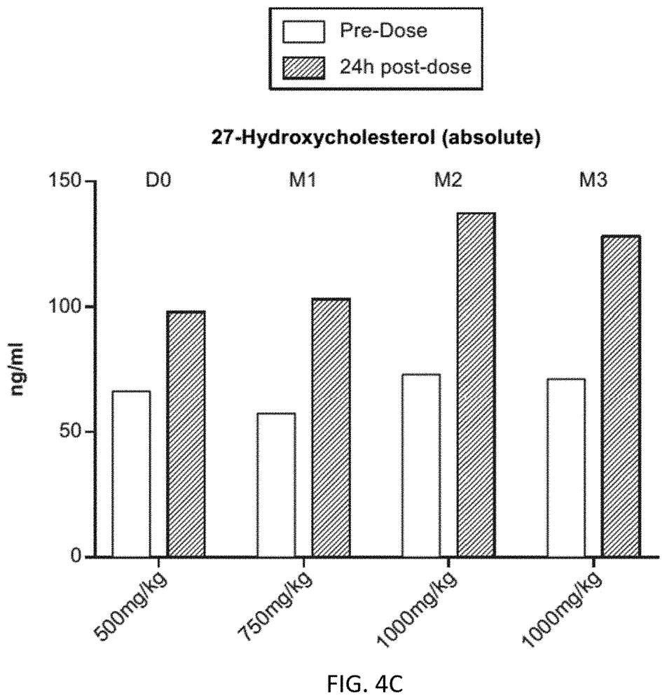

In this Example, a male human subject was treated with 2-hydroxypropyl-beta-cyclodextrin as in Example 2, and plasma levels of 24S-hydroxycholesterol and 27-hydroxycholesterol were measured. FIGS. 4A-4C demonstrate that treatment with 2-hydroxypropyl -beta-cyclodextrin led to increased plasma levels of 24S-hydroxycholesterol and 27-hydroxycholesterol, whereas total cholesterol levels remained stable. The increased production of 24S-hydroxycholesterol and 27-hydroxycholesterol enhance cholesterol metabolism, by acting as endogenous ligands for LXR transcription factors responsible for an anti-inflammatory gene signature. The elevated 27-hydroxycholesterol specifically is a cellular marker of macrophage activation and increased activity for cholesterol crystal phagocytosis and atherosclerotic plaque clearance and demonstrates that 2-hydroxypropyl-beta-cyclodextrin may be a suitable treatment for atherosclerosis and/or atherosclerotic cardiovascular disease.

### Example 4

### Treatment with 2-hydroxypropyl-beta-cyclodextrin Increases LXR Transcription Factor-Regulated Genes

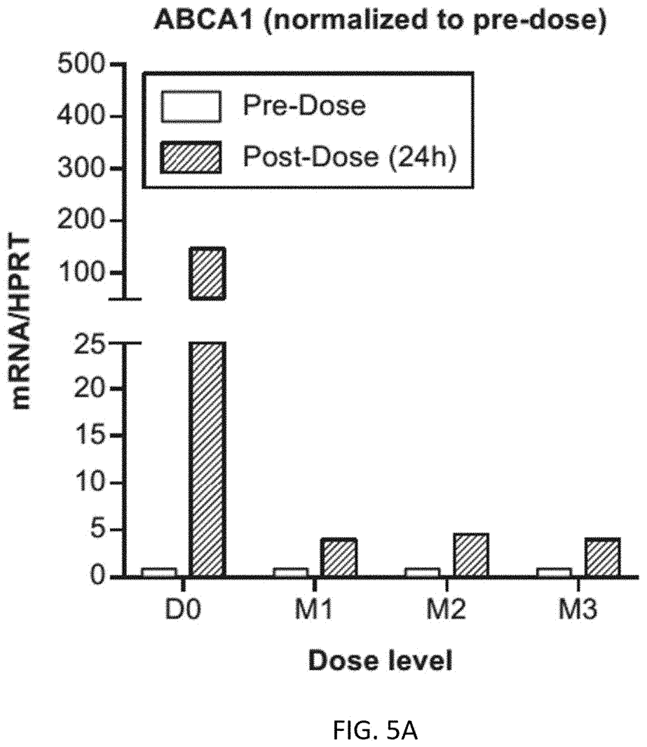

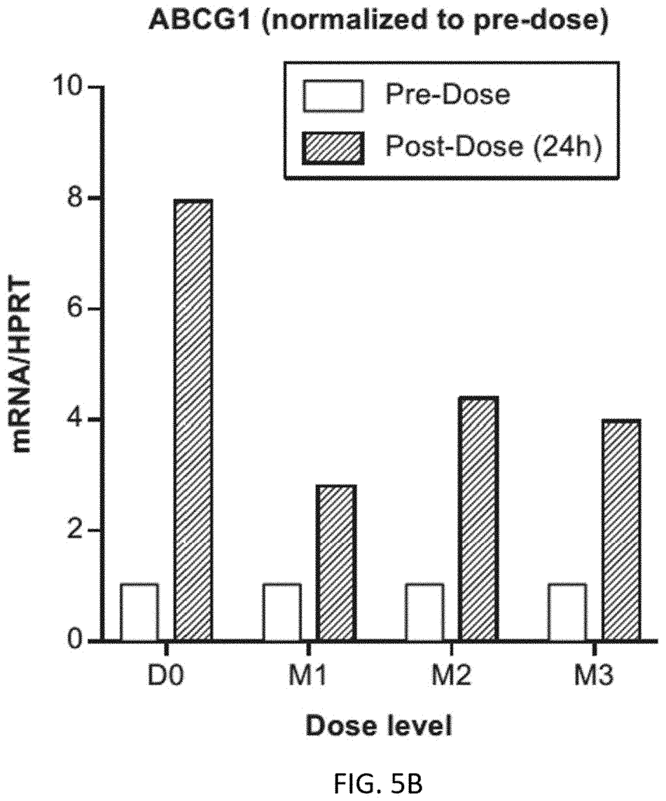

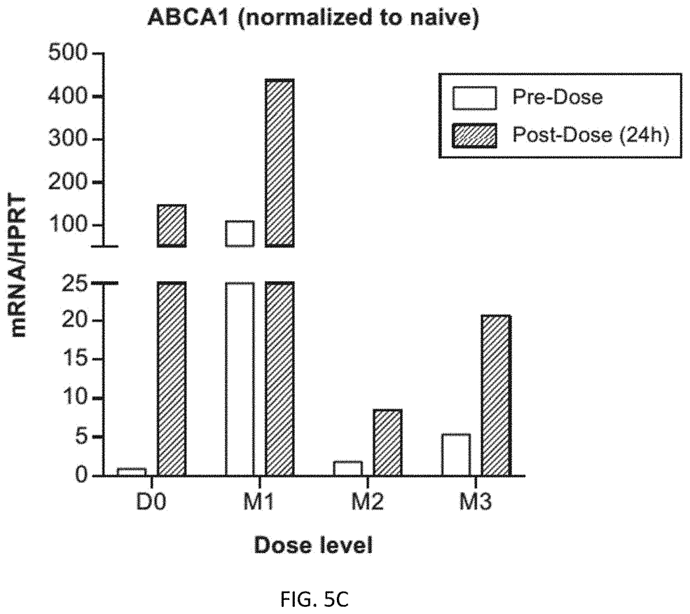

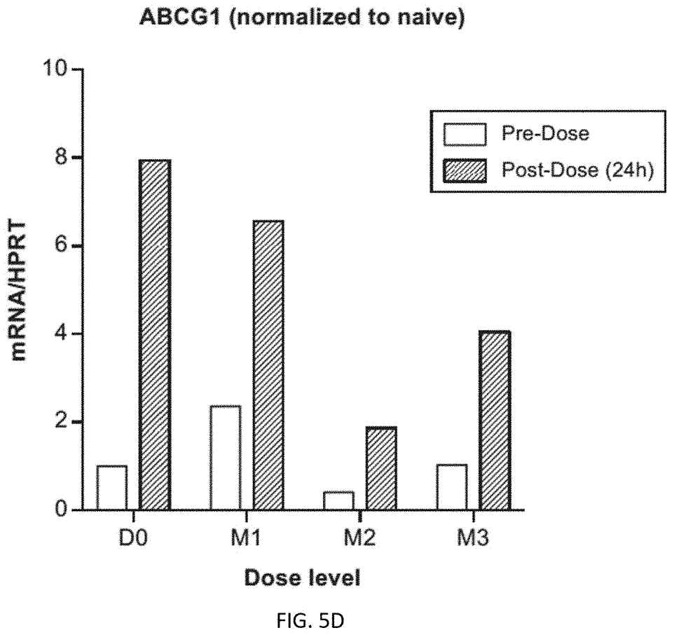

In this Example, a male human subject was treated with 2-hydroxypropyl-beta-cyclodextrin as in Example 2, and ABCA1 and ABCG1 mRNA levels were measured. FIGS. 5A-5D demonstrate that treatment with 2-hydroxypropyl-beta-cyclodextrin led to increased mRNA levels of LXR transcription factor-regulated genes ABCA1 and ABCG1. ABCA1 and ABCG1 are major cellular transporters resulting in cholesterol efflux to crude or mature HDL particles. In cardiovascular disease, expression of ABC transporters correlates with plaque stability and morbidity. This data suggests that activation of LXR transcription factor-regulated genes by administering 2-hydroxypropyl-beta-cyclodextrin may revert or reduce atherosclerotic plaque and may be a suitable for the treatment of atherosclerosis and/or atherosclerotic cardiovascular disease.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-47. (canceled)

48. A method of increasing plasma cholesterol crystal dissolution capacity (CCDC) in a human individual in need thereof, the method comprising: administering a therapeutically effective amount of 2-hydroxypropyl-beta-cyclodextrin (HPBCD) to the human individual, wherein the therapeutically effective amount of HPBCD is about 500 mg/kg to about 1,000 mg/kg.

49. The method of claim 48, wherein the therapeutically effective amount is about 4 g to about 250 g of HPBCD.

50. The method of claim 48, wherein the therapeutically effective amount of HPBCD is sufficient to achieve a serum, plasma, and/or whole blood concentration of HPBCD of about 0.6 mM to about 3 mM.

51. The method of claim 48, wherein the administering further comprises: (i) administering, at a first time point, a therapeutically effective first dose of HPBCD to the human individual; and (ii) administering, at a second time point, a therapeutically effective second dose of HPBCD to the human individual.

52. The method of claim 51, wherein the second time point is at least 1 week after the first time point.

53. The method of claim 48, wherein the administering is by intravenous administration.

54. The method of claim 48, wherein the therapeutically effective amount of HPBCD increases CCDC by at least about 10% as compared to prior to the administering.

55. The method of claim 48, wherein the therapeutically effective amount of HPBCD is an amount effective to
i) reduce or inhibit the development of cholesterol rich plaque;
ii) increase a circulating and/or systemic level of at least one oxysterol;
iii) increase a level of ABCA1 and/or ABCG1; or
iv) any combination thereof,
in the human individual.

56. The method of claim 55, wherein the at least one oxysterol in ii) is selected from the group consisting of: 27-hydroxycholesterol and 24-hydroxy cholesterol.

57. The method of claim 56, wherein the therapeutically effective amount is an amount effective to increase a circulating and/or systemic level of 27-hydroxycholesterol to at least about 100 ng/mL, or at least about 90 ng per mg of total circulating and/or systemic cholesterol.

58. The method of claim 55, wherein the therapeutically effective amount is an amount effective to increase a circulating and/or systemic level of at least one oxysterol to about 40 ng/mL or greater, or at least about 40 ng per mg of total circulating and/or systemic cholesterol.

59. The method of claim 55, wherein the therapeutically effective amount is an amount sufficient to sustain the circulating and/or systemic level of the at least one oxysterol for at least 24 hours.

60. The method of claim 55, wherein the therapeutically effective amount is an amount sufficient to sustain the circulating and/or systemic level of the at least one oxysterol for at least 48 hours.

61. The method of claim 48, wherein the human individual has or is in risk of atherosclerosis and/or atherosclerotic cardiovascular disease.

62. The method of claim 61, wherein the atherosclerotic cardiovascular disease is selected from the group consisting of: coronary artery disease (CAD), peripheral artery disease (PAD), and peripheral vascular disease (PVD).

63. The method of claim 61, wherein the therapeutically effective amount of HPBCD is an amount effective to decrease or prevent progression and/or development of atherosclerosis and/or atherosclerotic cardiovascular disease in the human individual.

64. The method of claim 48, wherein the administering results in at least one of the following in the human individual:
a) liver enzyme levels less than 2.5 times to normal;
b) serum creatinine levels less than 0.3 mg/dl; and
c) no substantial loss of sensorineural hearing.

65. The method of claim 48, wherein the administering results in a decrease in a risk of a major adverse cardiovascular event (MACE) in the human individual.

66. The method of claim 65, wherein the MACE comprises at least one of heart failure, re-infarction, recurrent angina pain, re-hospitalization for cardiovascular-related illness, repeat percutaneous coronary intervention (PCI), coronary artery bypass grafting, coronary revascularization, stroke, all-cause mortality (ACM), and a combination thereof.

67. A method of treating atherosclerosis and/or atherosclerotic cardiovascular disease in a human individual in need thereof, the method comprising: administering a therapeutically effective amount of 2-hydroxypropyl-beta-cyclodextrin (HPBCD) to the human individual, wherein the therapeutically effective amount of HPBCD is about 500 mg/kg to about 1,000 mg/kg.

68. The method of claim 67, wherein the atherosclerotic cardiovascular disease is selected from the group consisting of: coronary artery disease (CAD), peripheral artery disease (PAD), and peripheral vascular disease (PVD).

69. The method of claim 67, wherein the administering is by intravenous administration.

70. A pharmaceutical composition comprising about 500 mg/kg to about 1,000 mg/kg of 2-hydroxypropyl-beta-cyclodextrin (HPBCD) effective to increase plasma cholesterol crystal dissolution capacity (CCDC) in a human individual, and a pharmaceutically acceptable excipient.

71. A pharmaceutical composition comprising about 500 mg/kg to about 1,000 mg/kg of 2-hydroxypropyl-beta-cyclodextrin (HPBCD) effective to treat atherosclerosis and/or atherosclerotic cardiovascular disease in a human individual, and a pharmaceutically acceptable excipient.

72. The pharmaceutical composition of claim 70, formulated for single dose administration.

73. The pharmaceutical composition of claim 71, formulated for single dose administration.

74. The pharmaceutical composition of claim 70, formulated for intravenous administration.

75. The pharmaceutical composition of claim 71, formulated for intravenous administration.

76. A kit comprising:
(a) at least one container; and
(b) the pharmaceutical composition of claim 70, wherein the pharmaceutical composition is contained within the at least one container.

77. A kit comprising:
(a) at least one container; and
(b) the pharmaceutical composition of claim 71, wherein the pharmaceutical composition is contained within the at least one container.
